SEQUENCE LISTING (7 sequences)

SEQ ID NO: 1 - protein, 447 aa, Artificial
  Reconstructed human monoclonal antibody
EIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 217 aa, Artificial
  Reconstructed human monoclonal antibody
QVQLVESGGGVVQPGRSRRLSCAASGFTFSRYTMHWVRQAPGKGLEWVAVISFDGSNKYYVDSVKGRFTISRDNSENTLYLQVNILRAEDTAVYYCAREARGSYAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 3 - DNA, 18 bp, human
tgtctagaagctgggtac

SEQ ID NO: 4 - DNA, 35 bp, human
aagcggccgcctaaactttcttgtccaccttggtg

SEQ ID NO: 5 - DNA, 18 bp, human
tgtctagaagctgggtac

SEQ ID NO: 6 - DNA, 45 bp, human
gagcttcaacaggggagagtgtgagcccaaatcttgtgacaaaac

SEQ ID NO: 7 - DNA, 45 bp, human
gttttgtcacaagatttgggctcacactctcccctgttgaagctc

(19) United States
(12) Patent Application Publication
(10) Pub. No.: US 2006/0206947 A1
(43) Pub. Date: Sep. 14, 2006

(54) Heterodimeric protein binding compositions

(76) Inventor: Bernard J. Scallon, Wayne, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/361,528

(22) Filed: Feb. 24, 2006

Related U.S. Application Data
(60) Provisional application No. 60/657,050, filed on Feb. 28, 2005.

(51) Int. Cl. A01K 67/027; A01H 5/00; C12P 21/00; C07H 21/04; C12P 21/06; C12N 5/04; C12N 5/06; C07K 16/44

(52) U.S. Cl. 800/6; 435/69.1; 435/320.1; 435/326; 435/419; 530/387.3; 536/23.53; 800/288; 435/252.3; 435/254.2

(57) ABSTRACT

The invention relates to methods for making modified immunoglobulin compositions to maximize antigen binding and effector function by swapping of sequences of the heavy and light chains within an IgG Fab domain for the purpose of reorienting the Fc domain relative to the antigen-binding domain.

### BACKGROUND OF THE INVENTION

1. Field of the Invention

The present application claims the benefit of priority to U.S. Provisional Application Ser. No. 60/657,050, filed Feb. 28, 2005 the contents of which are completely incorporated by reference.

The invention relates to the field of heterodimeric protein binding compositions derived from immunoglobulin proteins, methods of preparing such heterodimeric protein binding compositions and uses thereof. More particularly, the invention relates to methods for making heterodimeric protein binding compositions to maximize immune effector functions, while preserving antigen binding, by swapping of sequences between the heavy and light chains within an IgG Fab domain for the purpose of reorienting the Fc domain relative to the antigen-binding domain and rendering critical binding sites more accessible.

2. Background and Related Art

For several decades antibodies have been indispensable in research and diagnosis and more recently in the therapeutic treatment of diseases due to their specific binding properties and high stability. Monoclonal antibodies were initially produced by fusing a chosen B cell line with an immortal myeloma cell line to produce hybridomas, immortal cells that secrete only the selected antibody type of the selected B cell clone. The use of recombinant DNA technologies has enabled new methods of producing antibodies as well as the design of new antibody constructs.

Structurally, each antibody is formed by the interaction of two identical “heavy” chains and two identical “light” chains, all of which combine to form a Y shape molecule (the heavy chains span the entire Y, and the light chains the two arms only). An immunoglobulin G antibody molecule contains complementary determining regions (CDRs) which determine antigen binding, constant regions that determine effector function and framework regions. Various constant regions can mediate a whole range of different biological effects. An allergic reaction, for example, follows IgE binding, whereas IgM binding can lead to the activation of the complement system.

Antibodies can be divided into five classes: IgG, IgM, IgA, IgD and IgE, based on the number of Y units and the type of heavy chain. The light chains of any antibody can be classified as either a kappa (k) or lambda (l) type (a description of molecular characteristics of the polypeptide); however, the heavy chain determines the subclass of each antibody. Heavy chains of IgG, IgM, IgA, IgD, and IgE, are known as gamma, mu, alpha, delta, and epsilon, respectively.

The most commonly used antibody is IgG, which can be cleaved into three parts, two F(ab) regions and one Fc, by the proteolytic enzyme papain, or into two parts, one F(ab′)2 and one Fc by the proteolytic enzyme pepsin. The F(ab) regions comprise the “arms” of the antibody, which are critical for antigen binding. The Fc region comprises the CH2 and CH3 domains of the constant region which makes up the “tail” of the antibody and plays a role in immune response, as well as serving as a useful “handle” for manipulating the antibody during some immunochemical procedures. The number of F(ab) regions on the antibody, corresponds with its subclass, and determines the “valency” of the antibody (loosely stated, the number of “arms” with which the antibody may bind its antigen).

Thus an antibody construct can include any protein or peptide containing molecule that comprises at least a portion of an immunoglobulin molecule, such as but not limited to at least one CDR of a heavy or light chain or a ligand binding portion thereof, a heavy chain or light chain variable region, a heavy chain or light chain constant region, a framework region, or any portion thereof. An antibody fragment can include the fragment of the immunoglobulin molecule known as the Fab containing the CDR antigen binding site, generated by cleavage of the antibody with the protease papain which cuts at the “hinge” region of the Y shaped antibody molecule producing two Fab fragments. An antibody can include or be derived from any mammal, such as but not limited to a human, a mouse, a rabbit, a rat, a rodent, a primate, or any combination thereof.

Numerous therapeutic IgG antibodies that bind cell-surface targets depend on simultaneous binding to Fc receptors (FcRs) on neighboring cells in order to realize their full therapeutic potential. This is because simultaneous engagement of FcRs and antigen, the means by which antibodies link cellular immune responses to humoral immune responses, can lead to effector functions such as antibody-dependent cell cytotoxicity (ADCC) or phagocytosis of the antigen-expressing cell. However, as more antibodies are getting evaluated in in vitro ADCC assays, it is becoming increasingly apparent that some antibodies may show good binding to a target (antigen-expressing) cell line and yet not trigger cytotoxicity of the target cells in the presence of FcR-expressing effector cells. Although one can envision other possible explanations, it is likely that, for at least some antibodies, the FcR binding site on such antibodies may not be physically accessible to FcRs on neighboring cells when these antibodies are bound to their cell-surface antigen (FIG. 1). Current data and models suggest that the Fc domain needs to bend approximately 90° relative to the Fab domains in order to accomodate FcR binding in the hinge region. For antibodies that are oriented “parallel” to the cell surface when bound to antigen, it may not be possible to assume this dramatic reconfiguration without steric hindrance from the plasma membrane or some other molecule, particularly if the antigen is rigid and incapable of “waving”. For the same reason, the C1q-binding site on antibodies may not be accessible to C1q, the first component of the classical complement fixation pathway, resulting in a lack of complement lysis activity.

IgG antibodies are flexible molecules, with the capacity to bend at several sites within the molecule, particularly the hinge region. Antibodies have also been reported to undergo a twisting/turning of the Fab domains relative to the Fc domains. Although this twisting/turning capability likely impacts the accessibility of the FcR-binding site to FcRs on neighboring cells, such mobility is limited and therefore unlikely to sufficiently render any FcR-binding site accessible to FcRs. Consequently, whereas a given antibody may have high affinity and specificity to a desired cell-surface antigen, its full therapeutic potential may still be limited if its orientation upon binding antigen does not favor recruitment of Fc-mediated effector functions. The invention described here offers a means of solving this problem for such antibodies.

### SUMMARY OF THE INVENTION

The invention is a heterodimeric protein binding composition having a heavy chain and a light chain of an immunoglobulin molecule, wherein the heterodimeric protein binding composition has heavy chain sequence replaced with light chain sequence and light chain sequence replaced with heavy chain sequence. Antibodies that bind to cell-surface (or otherwise insoluble) antigens in such a way as to have either their FcR-binding site inaccessible to FcRs on neighboring cells or C1q-binding site inaccessible to soluble C1q complement protein cannot take full advantage of their inherent ability to recruit effector functions such as ADCC, FcR-mediated phagocytosis, and complement lysis. The invention described here provides a solution to this problem by reorienting the relative position of the FcR-binding and C1q-binding domains relative to the antigen-binding domain. This is accomplished by swapping amino acid sequences between the heavy and light chains of the antibody (Ab) in a way that preserves the structure of the antigen-binding domain while orienting the rest of the Ab molecule in a very different position. Examples of sequences that could accomplish this by being swapped between heavy and light chains include a) the entire Fd domain (V_{H}-C_{H}1) of the heavy chain and the entire light chain (V_{L}-C_{L}), b) the variable region of the heavy chain (V_{H}) and the variable region of the light chain (V_{L}), and c) the complementarity-determining regions (CDRs) of V_{H} and the CDRs of V_{L}. Conversely, it may be desirable to modify an Ab whose FcR binding site and C1q binding site are readily accessible in order for these binding sites to be less accessible, e.g. so that immune effector functions are not triggered. This could be accomplished by the same types of sequence-swapping approaches described above.

Thus, in accordance with the invention, different subsets of sequences from the heavy and light chains of an immunoglobulin are swapped between heavy and light chains of a particular Ab for the purpose of reorienting the Fc domain relative to the antigen-binding domains. Appropriately-prepared Ab variants that have had these sequences swapped should retain antigen binding affinity and specificity but have their Fc domains “flipped” relative to the antigen. In the case of cell-surface antigen, this new orientation of the Fc domain may make the difference between poor accessibility to the FcR-binding site and good accessibility to the FcR-binding site of the Ab (FIG. 2B). Accessibility issues may be attributed to the Ab not being capable of assuming the necessary configuration to expose the FcR-binding site because of interference by the plasma membrane or adjacent molecules. Additionally, the invention allows for additional tailoring by mixing and matching Fc domains from different IgG isotypes (e.g. IgG1, IgG2, IgG3, or IgG4) or even different Ig classes (e.g. IgA, IgD, IgG, IgE, or IgM).

The present invention provides, in another aspect, isolated nucleic acid molecules comprising, a polynucleotide encoding the aforementioned heterodimeric protein binding constructs and at least one specified sequence, domain, portion or variant thereof. The present invention further provides recombinant vectors comprising such nucleic acid molecules, host cells containing such nucleic acids and/or recombinant vectors, and methods of making and/or using such nucleic acids, vectors and/or host cells.

The present invention also provides a method for expressing such heterodimeric protein binding constructs in a host cell, comprising culturing a host cell as described herein under conditions wherein at least one such heterodimeric protein binding construct is expressed in detectable and/or recoverable amounts. The host cell may be selected from COS-1, COS-7, HEK293, BHK21, CHO, BSC-1, Hep G2, Ag653, SP2/0, HeLa, myeloma, or lymphoma cells, or any derivative, immortalized or transformed cell thereof. Also provided is a method for producing at least one such heterodimeric protein binding construct, comprising translating the construct encoding nucleic acid under conditions in vitro, in vivo or in situ, such that the heterodimeric protein binding construct is expressed in detectable and/or recoverable amounts.

The present invention also provides at least one composition comprising both an isolated heterodimeric protein binding construct of the invention encoding nucleic acid and/or protein as described herein and a suitable carrier or diluent. The carrier or diluent may be pharmaceutically acceptable, according to known carriers or diluents. The composition may also comprise at least one further compound, protein or composition.

The present invention further provides at least one method or composition for administering a therapeutically effective amount of a heterodimeric protein binding construct of the invention to modulate or treat at least one disease condition in a cell, tissue, organ, animal or patient, prior to, subsequent to, or during a related condition, as known in the art and/or as described herein.

The present invention also provides at least one composition, device and/or method for the delivery of a therapeutically or prophylactically effective amount of at least one heterodimeric protein binding construct, according to the present invention.

The present invention further provides at least one heterodimeric protein binding construct method or composition, for diagnosing at least one disease condition in a cell, tissue, organ, animal or patient, prior to, subsequent to, or during a related condition, as known in the art and/or as described herein.

Also provided is a medical device, comprising at least one heterodimeric protein binding construct of the invention, wherein the device is suitable for contacting or administering the antibody construct by at least one mode selected from parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal.

Also provided is an article of manufacture for human pharmaceutical or diagnostic use, comprising packaging material and a container comprising a solution or a lyophilized form of at least one isolated heterodimeric protein binding construct of the present invention. The article of manufacture can optionally comprise having the container as a component of a parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal delivery device or system.

Also provided is a method for producing at least one isolated heterodimeric protein binding construct of the present invention, comprising a host, transgenic animal, transgenic plant or plant cell capable of expressing the antibody in recoverable amounts. Further provided in the present invention is at least one heterodimeric protein binding construct produced by the above method.

The present invention further provides any invention described herein.

### BRIEF DESCRIPTION OF THE FIGURES

In the drawings that form a portion of the specification:

FIG. 1 is a schematic depiction of how simultaneous binding by an Ab to cell-surface antigen and FcR on a neighboring cell can be impacted by the mobility of the antigen and the orientation of the Ab.

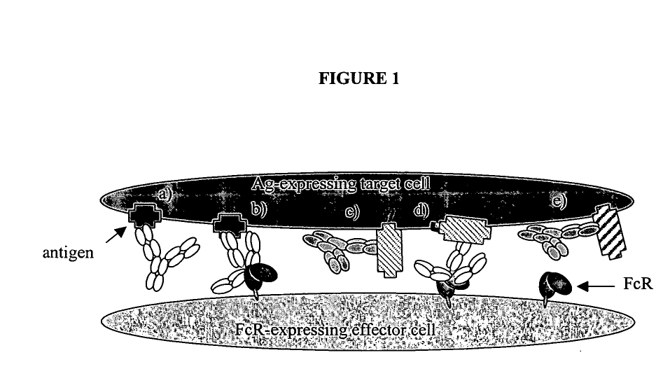

FIG. 2 is a schematic showing the effects of swapping sequences between heavy and light chains of an Ab. Fd=V_{H}-C_{H}1 domains of HC.

FIG. 3 shows how the relevant amino acid sequences of a unmodified Ab might compare to a modified Ab, in accordance with the invention. Amino acids are represented with single letter codes. Amino acids that originate from HC are shown in upper case letters and amino acids that originate from LC are shown in lower case letters. ‘X’ marks the point of chain cross-over.

FIG. 4 shows the binding data obtained by incubating cell supernatants from transfected cells containing either unmodified Ab or modified Ab with plates coated with mAbs (C508 or C585) specific for the antigen-binding region of the unmodified Ab, or a negative control Ab. Concentrations of the unmodified and modified Abs in the cell supernatants was previously determined using an anti-human Fc ELISA with purified unmodified Ab as standard.

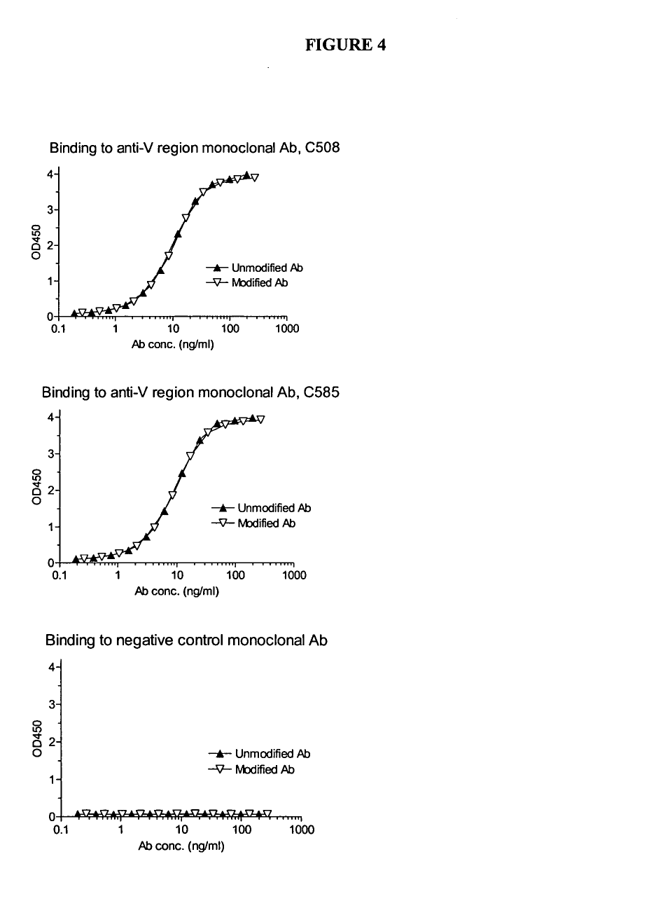

### DETAILED DESCRIPTION OF THE INVENTION

A. Definitions

Unless otherwise defined, scientific and technical terms used in connection with the present invention shall have the meanings that are commonly understood by those of ordinary skill in the art.

Furthermore, unless otherwise required by context, singular terms shall include pluralities and plural terms shall include the singular. Generally, nomenclatures utilized in connection with, and in techniques of, cell and tissue culture, molecular biology, protein and oligo- or polynucleotide chemistry and hybridization described herein are those well known and commonly used in the art. Standard techniques are used for recombinant DNA, oligonucleotide synthesis, tissue culture, and transformation (e.g., electroporation, lipofection).

Enzymatic reactions and purification techniques are performed according to the manufacturer's specifications, as commonly accomplished in the art, or as described herein. The foregoing techniques and procedures are generally performed according to conventional methods well known in the art and as described in various general and more specific references that are cited and discussed. (Seee.g., Sambrook et al. Molecular Cloning: A Laboratory Manual, 2^{nd} ed., Cold Spring Harbor Laboratory Press, Cold Spring Harbor, N.Y. 1989, which is incorporated herein by reference.) The nomenclatures utilized in connection the laboratory procedures and techniques of analytical, synthetic organic, medicinal and pharmaceutical chemistry, described herein, are those well known and commonly used in the art. Standard techniques are used for chemical syntheses, chemical analyses, pharmaceutical preparation, formulation, and the delivery and treatment of patients.

All scientific and technical terms used in this application have meanings commonly used in the art unless otherwise specified. As used in this application, the following words or phrases have the following meanings:

“Antibody”, “Ab” or “antibody peptide(s)” refers to an intact antibody, or a binding fragment thereof, that competes with the intact antibody for specific binding. Binding fragments are produced by recombinant DNA techniques, or by enzymatic or chemical cleavage of intact antibodies. Binding fragments include Fab,Fab′, F(ab1)2, Fv, and single-chain antibodies. An antibody other than a “bispecific” or “bifunctional” antibody is understood to have each of its binding sites identical. An antibody substantially inhibits adhesion of a receptor to a counter receptor when an excess of antibody reduces the quantity of receptor bound to counter receptor by at least about 20%, 40%,60% or 80%, and more usually, greater than about 85% (as measured in an in vitro competitive binding assay).

As used herein, “heterodimeric protein binding composition”; “modified Ig molecule”; or “swapped domain antibody construct” means an immunoglobulin (“Ig”) molecule that differs from a naturally-occurring Ig molecule in that the modified antibody has one or more heavy chain domains exchanged for domains on the light chain and/or one or more domains of the light chain exchanged for domains of the heavy chain; where the swapped domain may either be the same class or a different Ig class than the original antibody. A modified Ig molecule can be made, for example, by conventional genetic recombination using polynucleotides encoding Ig domains or portions thereof arranged in a chosen array and expressed in a cell. Alternatively, a modified Ig molecule can be synthesized using conventional techniques of polypeptide synthesis. The Ig molecule can be an IgA (which includes IgA1 and IgA2), IgM, IgG, IgD, or IgE molecule.

As used herein, “constant region domain” or “constant domain” refers to a domain within the constant portion of an Ig molecule, including C_{L}, C_{H}1, hinge, C_{H}2, C_{H} and C_{H}4. As used herein, a “variable region domain” or “variable domain” refers to that portion of an Ig molecule which confers specificity of the Ig for a particular antigen.

As used herein, “antigen” means a substance capable of either binding to an antigen binding region of an immunoglobulin molecule or of eliciting an immune response. As used herein, “antigen” includes, but is not limited to, antigenic determinants, haptens, and immunogens.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is 1 mM, preferably 100 nM and most preferably 10 nM.

As used herein, “vector” means a construct which is capable of delivering, and preferably expressing, one or more genes or polynucleotide sequences of interest in a host cell. Examples of vectors include, but are not limited to, viral vectors, naked DNA or RNA expression vectors, DNA or RNA expression vectors associated with cationic condensing agents, DNA or RNA expression vectors encapsulated in liposomes, and certain eucaryotic cells, such as producer cells.

As used herein, “polynucleotide” or “nucleic acid” means a deoxyribonucleotide or ribonucleotide polymer in either single- or double-stranded form, and unless otherwise limited, encompasses known analogs of natural nucleotides that hybridize to nucleic acids in a manner similar to naturally occurring nucleotides. Unless otherwise indicated, a particular nucleic acid sequence optionally includes the complementary sequence. The polynucleotide sequence may encode variable and/or constant region domains of immunoglobulin. The term “isolated polynucleotide” as used herein shall mean a polynucleotide of genomic, cDNA, or synthetic origin or some combination thereof. By virtue of its origin the “isolated polynucleotide” (1) is not associated with all or a portion of a polynucleotide in which the “isolated polynucleotides” are found in nature, (2) is operably linked to a polynucleotide which it is not linked to in nature, or (3) does not occur in nature as part of a larger sequence.

As used herein, “pharmaceutically acceptable carrier” includes any material which, when combined with an Ig, allows the Ig to retain biological activity and is non-reactive with the subject's immune system. Examples include, but are not limited to, any of the standard pharmaceutical carriers such as a phosphate buffered saline solution, water, emulsions such as an oil/water emulsion, and various types of wetting agents. Preferred diluents for aerosol or parenteral administration include phosphate buffered saline or normal (0.85%) saline.

As used in the appended claims, “a” means at least one, and can include a plurality. The term “operably linked” as used herein refers to positions of in a relationship permitting them to function in the intended manner. A control sequence “operably linked” to a coding sequence is ligated in such a way that expression of the coding sequence is achieved under conditions compatible with those of the control sequences.

The term “control sequence” as used herein refers to polynucleotide sequences which are necessary to effect the expression and processing of coding sequences to which they are ligated. The nature of such control sequences differs depending upon the host organism; in prokaryotes, such control sequences generally include a promoter, ribosomal binding site, and transcription termination sequence; in eukaryotes such control sequences generally include promoters and transcription termination sequences. The term “control sequences” is intended to include, at a minimum, all components whose presence is essential for expression and processing, and can also include additional components whose presence is advantageous, leader sequences and fusion partner sequences, for example.

As used herein, the twenty conventional amino acids and their abbreviations follow conventional usage. The amino acids that make up the modified antibodies of the present invention are often abbreviated. The amino acid designations can be indicated by designating the amino acid by its single letter code, its three letter code, name, or three nucleotide codon(s) as is well understood in the art (see Alberts, B., et al., Molecular Biology of The Cell, 3^{rd} Ed., Garland Publishing, Inc., New York, 1994).

As applied to polypeptides, the term “substantial identity” means that two peptide sequences, when optimally aligned, share at least 80% sequence identity, preferably at least 90% sequence identity, more preferably at least 95% sequence identity, and most preferably at least 99% sequence identity.

Preferably, residue positions which are not identical differ by conservative amino acid substitutions.

Conservative amino acid substitutions refer to the interchangeability of residues having similar side chains. For example: amino acids having aliphatic side chains are glycine, alanine, valine, leucine, and isoleucine; amino acids having aliphatic-hydroxyl side chains are serine and threonine; amino acids having amide-containing sidechains are asparagine and glutamine; amino acids having aromatic side chains are phenylalanine, tyrosine, and tryptophan; amino acids having basic side chains are lysine, arginine, and histidine; amino acids having sulfur-containing side chains are cysteine and methionine. Preferred-conservative amino acids substitution groups are: valine-leucine-isoleucine, phenylalanine-tyrosine, lysine-arginine, alanine-valine, glutamic-aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: aliphatic-hydrox=serine, threonine; amide-containing=asparagine, glutamine; aliphatic=alanine, valine, leucine, isoleucine; aromatic=phenylalanine, tryptophan, tyrosine. For example, it is reasonable to expect that an isolated replacement of a leucine with an isoleucine or valine, an aspartate with a glutamate, a threonine with aserine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specificactivity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains.

Structural and functional domains can be identified by comparison of the nucleotide and/or amino acid sequence data to public or proprietary sequence databases.

Preferably, computerized comparison methods are used to identify sequence motifs or predicted protein conformation domains that occur in other proteins of known structure and/or function. Methods to identify protein sequences that fold into a known three-dimensional structure are known. (Bowie et al. Science 253:164 (1991)). Thus, the foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of asequence other than the naturally occurring peptide sequence. For example, single or multiple amino acid substitutions (preferably conservative amino acid substitutions) may be made in the naturally occurring sequence (preferably in the portion of the polypeptide outside the domain(s) forming intermolecular contacts.

A conservative amino acid substitution should not substantially change the structural characteristics of the parent sequence (e.g., a replacement amino acid should not tend to break a helix that occurs in the parent sequence, or disrupt other types of secondary structures that characterize the parent sequence).

(Examples of art-recognized polypeptide secondary and S tertiary structures are described in Creighton, Ed., Proteins, Structures and Molecular Principles W.H. Freeman and Company, New York 1984; C. Branden and J. Tooze, eds.,Introduction to Protein Structure Garland Publishing, New York, N.Y. 1991; Thornton et at. Nature 354:105 1991, which are each incorporated herein by reference.)

The term patient includes human and veterinary subjects.

B. Antibody Structure

The basic antibody structural unit comprises a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chain constant regions are classified as μ, δ, γ, α, and ε, and define the antibody's isotype as IgM, IgD, IgG, IgA, and IgE, respectively.

Each of the gamma heavy chain constant regions contain CH1, hinge, CH2, and CH3 domains, with the hinge domain in gamma-3 being encoded by 4 different exons. (Morrison and Oi “Chimeric Ig Genes” in Immunoglobulin Genes pp. 259-274 Honjo et al. eds., Academic Press Limited, San Diego, Calif. 1989). Within light and heavy chains, the variable and constant regions are joined by a “J” region of about 12 or more amino acids, with the heavy chain also including a “D” region of about 10 more amino acids. (See generally: Fundamental Immunology Ch. 7 (Paul, W., ed., 2^{nd} ed. Raven Press, NY 1989) (incorporated by reference in its entirety for all purposes)). The variable regions of each light/heavy chain pair form the antibody binding site. Thus, an intact antibody has two binding sites. Except in bifunctional or bispecific antibodies, the two binding sites are the same. The chains all exhibit the same general structure of relatively conserved framework regions (FR) joined by three hyper variable regions, also called complementarity determining regions or CDRs.

The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health Bethesda, Md. 1987 and 1991; Chothia & Lesk J. Mol. Biol. 196:901-917 1987; Chothia et al. Nature 342:878-883 1989).

A bispecific or bifunctional antibody is an artificial hybrid antibody having two different heavy/light chain pairs and two different binding sites. Bispecific antibodies can be produced by a variety of methods including fusion of hybridomas or linking of Fab' fragments. (See, e.g., Songsivilai & Lachmann Clin. Exp. Immunol. 79:315-321 1990; Kostelny et al. J. Immunol. 148:1547-1553 1992).

Production of bispecific antibodies can be a relatively labor intensive process compared with production of conventional antibodies and yields and degree of purity are generally lower for bispecific antibodies.

Bispecific antibodies do not exist in the form of fragments having a single binding site (e.g., Fab, Fab and Fv).

C. Antibodies of the Present Invention

The present invention is specifically related to engineering of antibody molecules to create a modified antibody that has one or more heavy chain domains exchanged for domains on the light chain and/or one or more domains of the light chain exchanged for domains of the heavy chain for the purpose of reorienting the Fc domain relative to the antigen-binding domains.

In accordance with the present invention there are provided methods for the utilization of a plurality of native or modified immunoglobulin (Ig) constant domains to modify the characteristics of an antibody in its ability to interact with Fc receptors by swapping one or more immunoglobulin constant domains form the light and heavy chain in the constant region of the antibody. Standard recombinant DNA methods and/or DNA synthesis can be used by those skilled in the art to prepare genes encoding any combination of heavy and light chain sequences of a particular Ab once the amino acid sequence of that Ab has been determined. There are different subsets of sequences that might be swapped between heavy and light chains of a particular Ab for the purpose of reorienting the Fc domain relative to the antigen-binding domains. Examples include:

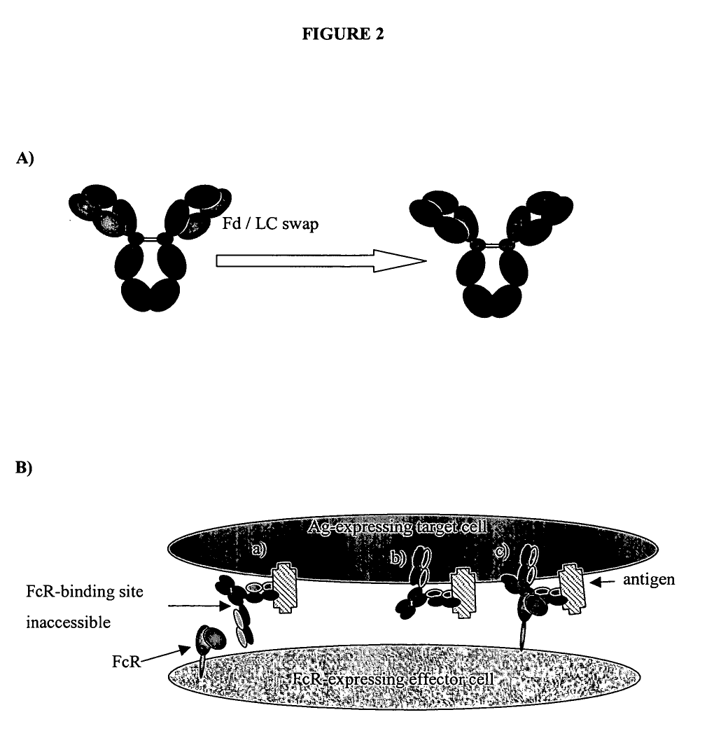

Heavy chain Fd and entire light chain (FIG. 2A)—making a V_{L}-C_{L}-hg-C_{H}2-C_{H}3 heavy chain and a V_{H}-C_{H}1 light chain should require the least amount of engineering. All points of contact between the Fd and light chain should be maintained.

V regions (FIG. 2A)—swapping the heavy and light chain V regions to make a V_{L}-C_{H}1-hg-C_{H}2-C_{H}3 heavy chain and a V_{H}-C_{L} light chain should only require minor engineering to accommodate new interfaces between the V domains and the adjacent C domains CDRs—these antigen-binding motifs on an Ab could theoretically be swapped between chains, although preserving antigen binding would likely require extensive engineering to the sequences flanking the CDRs.

Appropriately-prepared Ab variants that have had these sequences swapped should retain antigen binding affinity and specificity but have their Fc domains “flipped” relative to the antigen. In the case of cell-surface antigen, this new orientation of the Fc domain may make the difference between poor accessibility to the FcR-binding site and good accessibility to the FcR-binding site of the Ab (FIG. 2B). Accessibility issues may be attributed to the Ab not being capable of assuming the necessary configuration to expose the FcR-binding site because of interference by the plasma membrane or adjacent molecules.

Such Ab variants may also confer advantages against soluble antigens or viruses, e.g. large targets wherein the FcR-binding site or C1q-binding site is blocked by steric hindrance from a portion of the target other than the epitope. Inaccessibity of the FcR-binding site or C1q-binding site on such Abs could impact FcR-mediated clearance of immune complexes or complement-mediated lysis of the target.

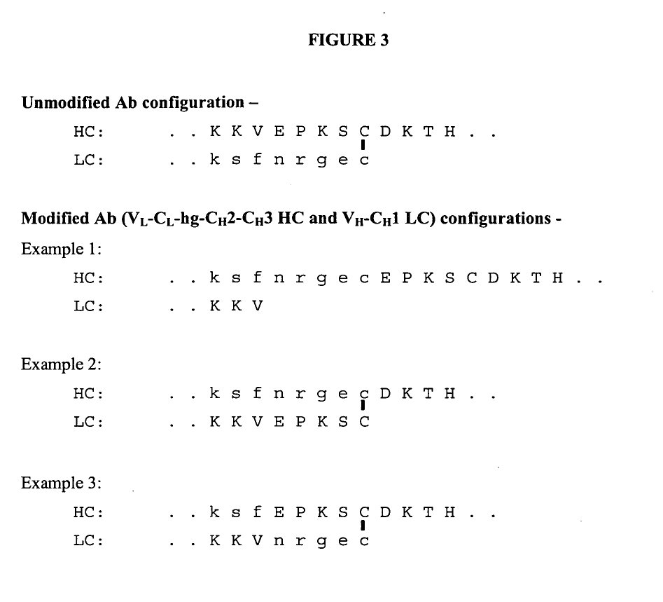

There will be some degree of flexibility in determining the exact point in the amino acid sequences to cross-over from one chain to the other. Two examples for V_{L}-C_{L}-hg-C_{H}2—C_{H}3 heavy chain and a V_{H}-C_{H}1 light chain are shown in FIG. 3.

An Ab that shows good binding to a tumor cell target but little ADCC activity would be a good candidate for this invention since a reorientation of the Fc domain could lead to enhanced ADCC activity against the tumor cells. The re-engineered genes could be prepared and then expressed in the same cell systems used for conventional Abs and the resulting Ab variants purified by the same methods used for conventional Abs, ie. protein A or protein G chromatography. After testing for antigen-binding capabilities, ADCC or complement lysis assays would indicate whether the re-engineered Ab variant had greater cell-killing activity than the original Ab. There may also be biophysical means of comparing accessibility of the Fc domain in the re-engineered vs original Ab, eg. by evaluating how well FcR⁺ cells bind to Ab that is bound to immobilized antigen.

The invention thus provides for a novel means to reorient, and possibly make much more accessible, the FcR-binding site and C1q-binding sites on IgG Abs. The efficacy of some Abs is likely to be dramatically enhanced by rendering these sites accessible, particularly Abs for whom ADCC, phagocytosis, or complement activation is a part of its mechanism of action. Still other Abs that do not recruit such immune effector functions as part of their mechanism of action may also benefit by this invention by realizing a higher avidity for its cell-surface or otherwise immobilized antigen due to the effects of simultaneous FcR binding. The greater the simultaneous FcR binding, the more likely it is that any Ab that momentarily dissociates from its antigen will be held by the FcR in close proximity to its antigen, thereby greatly increasing the chance of reassociation. This approach will often be a more attractive option than to search for a completely different Ab against the same antigen that binds in a more favorable orientation.

Physical linkage of the antibody domains may be accomplished utilizing any conventional technique. In preferred embodiments, physical linkage of the domains is accomplished recombinantly, i.e., wherein a gene construct encoding such domains is introduced into an expression system in a manner that allows correct assembly of the molecule upon expression therefrom. The foregoing example is depicted in FIGS. 2 and 3.

To construct such a modified Ig, in general, the DNA encoding selected HC or LC domains of the Ab to be modified can be readily isolated, engineered, and cloned into selected sites in the gene encoding the other chain of the same Ab. For instance, in one approach, the V_{H} and C_{H}1 coding sequences of a HC can be simultaneously PCR-amplified and modified to contain a translation stop codon immediately after the C_{H}1 coding sequence. The encoded polypeptide could constitute the LC of a modified Ab. At the same time, the V_{L} and C_{L} coding sequences for the same Ab can be simultaneously PCR-amplified and engineered to enable precise joining, e.g. by an overlapping PCR approach, of such coding sequence to sequence encoding the hinge, C_{H}2 and C_{H}3 domains of a HC. The polypeptide encoded by such a gene could constitute the HC of a modified Ab. These constructs for the light and heavy chains are then transfected into a suitable cell line for expression. In this manner, the molecule depicted in FIGS. 2B and 3 can be produced.

In the following examples, a sequence encoding a human V_{L} and C_{L} domain was inserted in place of the V_{H} and C_{H}1 domains on the heavy chain molecule of the same Ab. At the same time, the V_{H} and C_{H}1 domain was inserted in place of the V_{L} and C_{L} domains of the same Ab. Other preferred embodiments could include using V_{H} and C_{H}1 or the V_{L} and C_{L} coding sequences for another Ab, perhaps one that recognizes the same antigen as the original Ab or a different antigen. Such a strategy could alter antigen specificity, e.g. by bestowing greater reactivity to the corresponding antigen from other animal species, in addition to altering FcR binding. The swapped domain may also be a domain from a light chain of another Ig isotype such as IgD. Normally C_{H}1 domains of heavy chains are intimately associated with a light chain constant region and this association buries hybrophobic faces on both the heavy chain and the light chain.

Moreover, the swapped constant region need not be restricted to native forms of the constant regions that are present in native antibodies. Rather, the swapped constant region domain for use in accordance with the present invention can be generated through, for example, mutagenesis of constant region domains followed by screening for enhanced activity or prepared synthetically.

This invention could be practiced with antibodies from various species, such as humans, non-human primates, goats, rabbits, chickens, rats, hamsters, or mice. Other possibilities would be to insert an immunoglobulin domain from a non-Ab protein, such as CD4. The inserted sequence may not need to be an immunoglobulin domain. Other sequences may be able to confer the flexibility or spatial arrangement needed to improve Ab potency. Examples include the polypeptide linkers composed of glycine and serine residues, such as (Gly-Gly-Gly-Ser)₃.

It will be appreciated that the present invention is also applicable to enhancing the interactions between a receptor and its ligand generally. In this respect, either receptor or ligand moieties may be modified so as to generate molecules that possess greater than one moiety that enhances the affinity, avidity, or simply the ability of receptor and ligand to interact. Stated another way, the invention, by modifying the spatial characteristics of the Fc receptor binding domains, provides a method to increase avidity of a molecule to its Fc target. The end result is that the modified molecule will have a higher affinity for Fc receptors. In addition, because swapping immunoglobulin domains does not introduce foreign protein sequences the modified molecules are less likely to be immunogenic.

E. Design of Modified Antibodies

As discussed above, the basic design used to prepare a preferred modified antibody construct in accordance with the present invention is to substitute one or more heavy chain domains into the light chain constant region of an antibody and/or substitute one or more light chain domains into the heavy chain constant region. One construct in accordance with the invention is swapping the heavy and light chain V regions to make a V_{L}-C_{H}1-hg-C_{H}2-C_{H}3 heavy chain and a V_{H}-C_{L} light chain (as shown in FIG. 2A). The antibody which is to be modified may be selected from any antibody of human, rodent or other source, and may be a chimeric, humanized, human or synthetic antibody. In one embodiment, the antibody which is to be modified may be generated through immunization of a normal or transgenic mouse. The antibody may be further modified in any of a number of ways known in the art. In general the modified antibody may be prepared by simply substituting the polynucleotide encoding the swapped constant domain or other insert sequence into the plasmid encoding the constant region of the antibody and expressing the plasmid in a suitable host cell to produce the modified antibody. The insert may be made directly or with a linker molecule. The nature of the insert and linker can be designed as necessary to perform the function intended, i.e. to modify the Fc receptor binding of the antibody molecule. The amino acid composition and length of the insert modifying the antibody immunoglobulin molecule may be determined by testing constructs containing a variety of different sequences as known in the art.

Where a modified molecule that has certain characteristics is desired, it may be desirable or necessary to introduce certain mutations in the constant region insert so as to modify its characteristics in some way. However, where an antibody for use in humans is desired, it is desirable to make the inserts as close to human sequences as possible to reduce immunogenicity. Accordingly, it is generally desirable to introduce as few amino-acid changes to the modified molecules as possible so as to avoid generating immunogenicity.

Bispecific, heterospecific, heteroconjugate or similar monoclonal, humanized antibodies that have binding specificities for at least two different antigens can also be used. In such a case, one of the binding specificities may be designated for one antigen and the other one is for any other antigen. Methods for making bispecific antibodies are known in the art. Traditionally, the recombinant production of bispecific antibodies is based on the co-expression of two immunoglobulin heavy chain/light chain pairs, where the two heavy chains have different specificities (Milstein and Cuello, Nature 305:537 1983). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed, (e.g., WO 93/08829, U.S. Pat. Nos. 6,210,668, 6,193,967, 6,132,992, 6,106,833, 6,060,285, 6,037,453, 6,010,902, 5,989,530, 5,959,084, 5,959,083, 5,932,448, 5,833,985, 5,821,333, 5,807,706, 5,643,759, 5,601,819, 5,582,996, 5,496,549, 4,676,980, WO 91/00360, WO 92/00373, EP 03089, Traunecker et al., EMBO J. 10:3655 1991; Suresh et al., Methods in Enzymology 121:210 1986, each entirely incorporated herein by reference).

In the following examples, the modified Ab was prepared by using recombinant DNA methods to substitute the DNA sequence encoding the heavy chain Fd region to make a V_{L}-C_{L}1-hg-C_{H}2-C_{H}3 heavy chain and a V_{H}-C_{H}1-hg-C_{L}2-C_{L}3 light chain.

The sequences for the modified antibody compared to the unmodified murine antibody from which it was derived, are shown in FIG. 3.

Preferably, the modified antibody construct or ligand-binding portion or variant thereof binds at least one protein ligand or receptor, and thereby provides at least one biological activity of the corresponding protein or a fragment thereof. Different therapeutically or diagnostically significant proteins are well known in the art and suitable assays or biological activities of such proteins are also well known in the art. Modified antibodies that bind any number of biologically active proteins may be used in conjunction with the present invention. Of particular interest are antibodies that bind to, and thus modulate the activity of TNF, leptin, any of the interleukins (IL-1 through IL-23, etc.), and proteins involved in complement activation (e.g., C3b). Targeting proteins that are differentially expressed in certain disease states are also of interest, including proteins expressed on tumors and the like. All of these classes of ligands may be discovered by methods described in the references cited in this specification and other references. A particularly preferred group of modified antibodies are those that bind to cytokine receptors. Cytokines have recently been classified according to their receptor code (see Inglot 1997, Archivum Immunologiae Therapiae Experimentalis 45: 353-7, which is hereby incorporated entirely by reference).

Modified antibodies of the invention that comprise a modified constant region can be prepared using antibodies derived from any suitable methods, such as hybridomas, phage display (Katsube, Y., et al., Int J. Mol. Med, 1(5):863-868 1998) or methods that employ transgenic animals, as known in the art and/or as described herein.

A modified antibody construct of the present invention can include one or more amino acid substitutions, deletions or additions, either from natural mutation or human manipulation, from the parent antibody from which it was derived.

In another aspect, the modified antibody construct, as described herein, may be further modified by the covalent attachment of an organic moiety. Such modification can produce an antibody or antigen-binding fragment with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified antibodies and antigen-binding fragments of the invention can comprise one or more organic moieties that are covalently bonded, directly or indirectly, to the antibody. Each organic moiety that is bonded to an antibody or antigen-binding fragment of the invention can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, an antibody modified by the covalent attachment of polylysine is encompassed by the invention. Hydrophilic polymers suitable for modifying antibodies of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the antibody of the invention has a molecular weight of about 800 to about 150,000 Daltons as a separate molecular entity. For example PEG₅₀₀₀ and PEG_{20,000}, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying antibodies of the invention can be saturated or can contain one or more units of unsaturation. Fatty acids that are suitable for modifying antibodies of the invention include, for example, n-dodecanoate (C₁₂, laurate), n-tetradecanoate (C₁₄, myristate), n-octadecanoate (C₁₈, stearate), n-eicosanoate (C₂₀, arachidate), n-docosanoate (C₂₂, behenate), n-triacontanoate (C₃₀), n-tetracontanoate (C₄₀), cisα α9-octadecanoate (C₁₈, oleate), all cisα5,8,11,14-eicosatetraenoate (C₂₀, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably one to about six, carbon atoms.

The modified human antibodies and antigen-binding fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups such as tosylate, mesylate, halo (chloro, bromo, fluoro, iodo), N-hydroxysuccinimidyl esters (NHS), and the like. Activating groups that can react with thiols include maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see e.g., Hermanson, G. T., Bioconjugate Techniques, Academic Press San Diego, Calif. 1996). An activating group can be bonded directly to the organic group (e.g., hydrophilic polymer, fatty acid, fatty acid ester), or through a linker moiety, for example a divalent C₁-C₁₂ group wherein one or more carbon atoms can be replaced by a heteroatom such as oxygen, nitrogen or sulfur. Suitable linker moieties include, for example, tetraethylene glycol, —(CH₂)₃—, —NH—(CH₂)₆—NH—, —(CH₂)₂—NH— and —CH₂—O—CH₂—CH₂—O—CH₂—CH₂—O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate as described, or can be reacted with maleic anhydride and the resulting product cyclized to produce an activated maleimido derivative of the fatty acid. (See e.g., Thompson, et al., WO 92/16221;

the entire teachings of which are incorporated herein by reference.) The modified antibodies of the invention can be produced by reacting a human antibody or antigen-binding fragment with a modifying agent. For example, the organic moieties can be bonded to the antibody in a non-site specific manner by employing an amine-reactive modifying agent, for example, an NHS ester of PEG. Modified human antibodies or antigen-binding fragments can also be prepared by reducing disulfide bonds (e.g., intra-chain disulfide bonds) of an antibody or antigen-binding fragment. The reduced antibody or antigen-binding fragment can then be reacted with a thiol-reactive modifying agent to produce the modified antibody of the invention. Modified human antibodies and antigen-binding fragments comprising an organic moiety that is bonded to specific sites of an antibody of the present invention can be prepared using suitable methods, such as reverse proteolysis (Fisch et al., Bioconjugate Chem., 3:147-153 1992; Werlen et al., Bioconjugate Chem., 5:411-417 1994; Kumaran et al., Protein Sci. 6(10):2233-2241 1997; Itoh et al., Bioorg. Chem., 24(1): 59-68 1996; Capellas et al., Biotechnol. Bioeng., 56(4):456-463 1997; and the methods described in Hermanson, G. T., Bioconjugate Techniques, Academic Press San Diego, Calif. 1996)

F. Preparation of Modified Antibodies

Human genes which encode the constant (C) regions of the chimeric antibodies, fragments and regions of the present invention can be derived from a human fetal liver library by known methods. Human C region genes can be derived from any human cell including those which express and produce human immunoglobulins. The human CH region can be derived from any of the known classes or isotypes of human H chains, including γ, μ, α, δ, ε, and subtypes thereof, such as G1, G2, G3 and G4. Since the H chain isotype is responsible for the various effector functions of an antibody, the choice of C_{H} region will be guided by the desired effector functions, such as complement fixation, or activity in antibody-dependent cellular cytotoxicity (ADCC). Preferably, the C_{H} region is derived from γ1 (IgG1). The human C_{L} region can be derived from either human L chain isotype, κ or λ, preferably κ.

Genes encoding human immunoglobulin C regions are obtained from human cells by standard cloning techniques (Sambrook, et al. Molecular Cloning: A Laboratory Manual,2^{nd} Edition, Cold Spring Harbor Press, Cold Spring Harbor, N.Y. 1989; Ausubel et al, eds. Current Protocols in Molecular Biology 1987-1993). Human C region genes are readily available from known clones containing genes representing the two classes of L chains, the five classses of H chains and subclasses thereof. Chimeric antibody fragments, such as F(ab¹)₂ and Fab, can be prepared by designing a chimeric H chain gene which is appropriately truncated. For example, a chimeric gene encoding an H chain portion of an F(ab¹)₂ fragment would include DNA sequenes encoding the CH1 domain and hinge region of the H chain, followed by a translational stop codon to yield the truncated molecule.

Generally, the murine, human or murine and chimeric antibodies, fragments and regions of the present invention are produced by cloning DNA segments encoding the H and L chain antigen-binding regions of a specific antibody, and joining these DNA segments to DNA segments encloding C_{H} and C_{L} regions, respectively, to produce murine, human or chimeric immunoglobulin-encoding genes.

Thus, in a preferred embodiment, a fused chimeric gene is created which comprises a first DNA segment that encodes at least the antigen-binding region of an antibody of human or non-human origin, such as a functionally rearranged V region with joining (J) segment, linked to a second DNA segment encoding at least a part of a human C region containing the swapped sequence.

The sequences of the variable, constant or insert sequence, may be modified by insertions, substitutions and deletions to the extent that the chimeric antibody maintains the ability to bind to and inhibit the antigen of interest. The ordinarily skilled artisan can ascertain the maintenance of this activity by performing the functional assays applicable.

The antibody construct of the present invention can be optionally produced by a cell line, a mixed cell line, an immortalized cell or clonal population of immortalized cells, as well known in the art. (See, e.g., Ausubel, et al., ed., Current Protocols in Molecular Biology, John Wiley & Sons, Inc., NY, N.Y. 1987-2001; Sambrook, et al., Molecular Cloning: A Laboratory Manual, 2^{nd} Edition, Cold Spring Harbor, N.Y. 1989; Harlow and Lane, antibodies, a Laboratory Manual, Cold Spring Harbor, N.Y. 1989; Colligan, et al., eds., Current Protocols in Immunology, John Wiley & Sons, Inc., NY 1994-2001; Colligan et al., Current Protocols in Protein Science, John Wiley & Sons, NY, N.Y. 1997-2001, each entirely incorporated herein by reference.)

In one approach, a hybridoma is produced by fusing a suitable immortal cell line (e.g., a myeloma cell line such as, but not limited to, Sp2/0, Sp2/0-AG14, NS/O, NS1, NS2, AE-1, L.5, >243, P3X63Ag8.653, Sp2 SA3, Sp2 MAI, Sp2 SS1, Sp2 SA5, U937, MLA 144, ACT IV, MOLT4, DA-1, JURKAT, WEHI, K-562, COS, RAJI, NIH 3T3, HL-60, MLA 144, NAMAIWA, NEURO 2A, or the like, or heteromylomas, fusion products thereof, or any cell or fusion cell derived therefrom, or any other suitable cell line as known in the art. (See, e.g., www.atcc.org, www.lifetech.com.), and the like, with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. (See, e.g., Ausubel, supra, and Colligan, Immunology, supra, chapter 2, entirely incorporated herein by reference.)

Any other suitable host cell can also be used for expressing heterologous or endogenous nucleic acid encoding a modified antibody, specified fragment or variant thereof, of the present invention. The fused (hybridomas) or recombinant cells can be isolated using selective culture conditions or other suitable known methods, and cloned by limiting dilution, cell sorting, or other known methods. Cells which produce antibodies with the desired specificity can be selected by a suitable assay (e.g., ELISA).

Methods for engineering or humanizing non-human or human antibodies can be used and are well known in the art. Generally, a humanized or engineered antibody has one or more amino acid residues from a source which is non-human. These human amino acid residues are often referred to as “import” residues, which are typically taken from an “import” variable, constant or other domain of a known human sequence. Known human Ig sequences are disclosed; (e.g., www.ncbi.nlm.nih.gov/entrez/query.fcgi;

www.atcc.org/phage/hdb.html; www.sciquest.com/; www.abcam.com/;

www.antibodyresource.com/onlinecomp.html;

www.public.iastate.edu/˜pedro/research_tools.html; www.mgen.uni-heidelberg.de/SD/IT/IT.html; www.whfreeman.com/immunology/CH05/kuby05.htm;

www.library.thinkquest.org/12429/Immune/Antibody.html;

www.hhmi.org/grants/lectures/1996/vlab/; www.path.cam.ac.uk/˜mrc7/mikeimages.html; www.antibodyresource.com/;

mcb.harvard.edu/BioLinks/Immunology.html.www.immunologylink.com/;

pathbox.wustl.edu/˜hcenter/index.html; www.biotech.ufl.edu/˜hcl/;

www.pebio.com/pa/340913/340913.html; www.nal.usda.gov/awic/pubs/antibody/;

www.m.ehime-u.ac.jp/˜yasuhito/Elisa.html; www.biodesign.com/table.asp;

www.icnet.uk/axp/facs/davies/links.html; www.biotech.ufl.edu/˜fccl/protocol.html;

www.isac-net.org/sites_geo.html; aximtl.imt.uni-marburg.de/˜rek/AEPStart.html;

baserv.uci.kun.nl/˜jraats/linksl.html; www.recab.uni-hd.de/immuno.bme.nwu.edu/;

www.mrc-cpe.cam.ac.uk/imt-doc/public/INTRO.html; www.ibt.unam.mx/vir/V_mice.html;

imgt.cnusc.fr:8104/; www.biochem.ucl.ac.uk/˜martin/antibodies/index.html;

antibody.bath.ac.uk/;

abgen.cvm.tamu.edu/lab/wwwabgen.html;

www.unizh.ch/˜honegger/AHOseminar/Slide01.html; www.cryst.bbk.ac.uk/˜ubcg07s/;

www.nimr.mrc.ac.uk/CC/ccaewg/ccaewg.htm;

www.path.cam.ac.uk/˜mrc7/humanisation/TAHHP.html;

www.ibt.unam.mx/vir/structure/stat_aim.html;

www.biosci.missouri.edu/smithgp/index.html;

Kabat et al., Sequences of Proteins of Immunological Interest, U.S. Dept. Health 1983; each entirely incorporated herein by reference.)

Such imported sequences can be used to reduce immunogenicity or reduce, enhance or modify binding, affinity, on-rate, off-rate, avidity, specificity, half-life, or any other suitable characteristic, as known in the art. Generally part or all of the non-human or human CDR sequences are maintained while the non-human sequences of the variable and constant regions are replaced with human or other amino acids. Antibodies can also be humanized with retention of high affinity for the antigen and other favorable biological properties. To achieve this goal, humanized antibodies can be optionally prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding. Humanization or engineering of antibodies of the present invention can be performed using any known method, such as but not limited to those described in: (Winter, Jones et al., Nature 321:522 1986; Riechmann et al., Nature 332:323 1988; Verhoeyen et al., Science 239:1534 1988; Sims et al., J. Immunol. 151: 2296 1993; Chothia and Lesk, J. Mol. Biol. 196:901 1987; Carter et al., Proc. Natl. Acad. Sci. U.S.A. 89:4285 1992; Presta et al., J. Immunol. 151:2623 1993; U.S. Pat. Nos. 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539; 4,816,567, PCT/: US98/16280, US96/18978, US91/09630, US91/05939, US94/01234, GB89/01334, GB91/01134, GB92/01755; WO90/14443, WO90/14424, WO90/14430, EP 229246, each entirely incorporated herein by reference, included references cited therein.)

Antibodies of the present invention can also be prepared using at least one antibody construct encoding nucleic acid to provide transgenic animals or mammals, such as goats, cows, horses, sheep, and the like, that produce such antibodies in their milk. Such animals can be provided using known methods. (See, e.g., but not limited to, U.S. Pat. Nos: 5,827,690; 5,849,992; 4,873,316; 5,849,992; 5,994,616, 5,565,362; 5,304,489, and the like, each of which is entirely incorporated herein by reference.)

Antibodies of the present invention can additionally be prepared using at least one antibody construct encoding nucleic acid to provide transgenic plants and cultured plant cells (e.g., but not limited to tobacco and maize) that produce such antibodies, specified portions or variants in the plant parts or in cells cultured therefrom. As a non-limiting example, transgenic tobacco leaves expressing recombinant proteins have been successfully used to provide large amounts of recombinant proteins, e.g., using an inducible promoter. (See, e.g., Cramer et al., Curr. Top. Microbol. Immunol. 240:95-118 1999) and references cited therein. Also, transgenic maize has been used to express mammalian proteins at commercial production levels, with biological activities equivalent to those produced in other recombinant systems or purified from natural sources. (See, e.g., Hood et al., Adv. Exp. Med. Biol. 464:127-147 1999 and references cited therein.) Antibodies have also been produced in large amounts from transgenic plant seeds including antibody fragments, such as single chain antibodies (scFv's), including tobacco seeds and potato tubers. (See, e.g., Conrad et al., Plant Mol. Biol. 38:101-109 1998 and reference cited therein.) Thus, antibodies of the present invention can also be produced using transgenic plants, according to know methods. (See also, e.g., Fischer et al., Biotechnol. Appl. Biochem. 30:99-108 October, 1999: Ma et al., Trends Biotechnol. 13:522-7 199; Ma et al., Plant Physiol. 109:341-6 1995; Whitelam et al., Biochem. Soc. Trans. 22:940-944 1994; and references cited therein; each of the above references is entirely incorporated herein by reference.)

The affinity or avidity of an antibody for an antigen can be determined experimentally using any suitable method. (See, for example, Berzofsky, et al., “Antibody-Antigen Interactions,” In Fundamental Immunology, Paul, W. E., Ed., Raven Press NY, N.Y. 1984; Kuby, Janis Immunology, W. H. Freeman and Company NY, N.Y. 1992; and methods described herein.) The measured affinity of a particular antibody-antigen interaction can vary if measured under different conditions (e.g., salt concentration, pH). Thus, measurements of affinity and other antigen-binding parameters (e.g., K_{D}, Kₐ, K_{d}) are preferably made with standardized solutions of antibody and antigen, and a standardized buffer, such as the buffer described herein.

G. Nucleic Acid Molecules

Using the information provided herein, a nucleic acid molecule of the present invention encoding an antibody construct of the invention can be obtained using methods described herein or as known in the art.

Nucleic acid molecules of the present invention can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules of the present invention can include nucleic acid molecules comprising an open reading frame (ORF), optionally with one or more introns, at least one specified portion of at least one CDR, as CDR1, CDR2 and/or CDR3 of at least one heavy chain or light chain nucleic acid molecules comprising the coding sequence for a modified antibody construct and nucleic acid molecules which comprise a nucleotide sequence substantially different from those described above but which, due to the degeneracy of the genetic code, still encode at least one such modified antibody construct as described herein and/or as known in the art. Of course, the genetic code is well known in the art. Thus, it would be routine for one skilled in the art to generate such degenerate nucleic acid variants that code for specific antibodies of the present invention. (See, e.g., Ausubel, et al., supra), and such nucleic acid variants are included in the present invention.

As indicated herein, nucleic acid molecules of the present invention which comprise a nucleic acid encoding an antibody construct can include, but are not limited to, those encoding the amino acid sequence of an antibody fragment by itself, the coding sequence for the entire antibody or a portion thereof, the coding sequence for an antibody, fragment or portion, as well as additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example—ribosome binding and stability of mRNA); an additional coding sequence that codes for additional amino acids, such as those that provide additional functionalities. Thus, the sequence encoding an antibody can be fused to a marker sequence, such as a sequence encoding a peptide that facilitates purification of the fused antibody comprising an antibody fragment or portion.

Construction of Nucleic Acids

The isolated nucleic acids of the present invention can be made using (a) recombinant methods, (b) synthetic techniques, (c) purification techniques, or combinations thereof, as well-known in the art.

The nucleic acids can conveniently comprise sequences in addition to a polynucleotide of the present invention. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the present invention. For example, a hexa-histidine marker sequence provides a convenient means to purify the proteins of the present invention. The nucleic acid of the present invention—excluding the coding sequence—is optionally a vector, adapter, or linker for cloning and/or expressing a polynucleotide of the present invention.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra)

. Recombinant Methods for Constructing Nucleic Acids

The isolated nucleic acid compositions of this invention, such as RNA, cDNA, genomic DNA, or any combination thereof, can be obtained from biological sources using any number of cloning methodologies known to those of skill in the art. In some embodiments, oligonucleotide probes that selectively hybridize, under stringent conditions, to the polynucleotides of the present invention are used to identify the desired sequence in a cDNA or genomic DNA library. The isolation of RNA, and construction of cDNA and genomic libraries, is well known to those of ordinary skill in the art. (See, e.g., Ausubel, supra; or Sambrook, supra)

Nucleic Acid Screening and Isolation Methods

A cDNA or genomic library can be screened using a probe based upon the sequence of a polynucleotide of the present invention, such as those disclosed herein. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by temperature, ionic strength, pH and the presence of a partially denaturing solvent such as formamide. For example, the stringency of hybridization could be conveniently varied by changing the polarity of the reactant solution through manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the present invention without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; U.S. Pat. Nos. 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (see, e.g., Ausubel, supra; Sambrook, supra; U.S. Pat. No. 5,130,238 to Malek, et al, with the tradename NASBA; the entire contents of which references are incorporated herein by reference).

For instance, polymerase chain reaction (PCR) technology can be used to amplify the sequences of polynucleotides of the present invention and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. (Examples of techniques sufficient to direct persons of skill through in vitro amplification methods are found in: Berger, supra; Sambrook, supra; Ausubel, supra; Mullis, et al., U.S. Pat. No. 4,683,202 1987; Innis, et al., PCR Protocols A Guide to Methods and Applications, Eds., Academic Press Inc., San Diego, Calif. 1990.) Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

Synthetic Methods for Constructing Nucleic Acids

The isolated nucleic acids of the present invention can also be prepared by direct chemical synthesis using known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

H. Recombinant Expression Cassettes

The present invention further provides recombinant expression cassettes comprising a nucleic acid of the present invention. A nucleic acid sequence of the present invention, for example a cDNA or a genomic sequence encoding an antibody of the present invention, can be used to construct a recombinant expression cassette that can be introduced into at least one desired host cell. A recombinant expression cassette will typically comprise a polynucleotide of the present invention operably linked to transcriptional initiation regulatory sequences that will direct the transcription of the polynucleotide in the intended host cell. Both heterologous and non-heterologous (i.e., endogenous) promoters can be employed to direct expression of the nucleic acids of the present invention.

In some embodiments, isolated nucleic acids that serve as promoter, enhancer, or other elements can be introduced in the appropriate position (upstream, downstream or in intron) of a non-heterologous form of a polynucleotide of the present invention so as to up or down regulate expression of a polynucleotide of the present invention. For example, endogenous promoters can be altered in vivo or in vitro by mutation, deletion and/or substitution.

I. Vectors And Host Cells

The present invention also relates to vectors that include isolated nucleic acid molecules of the present invention, host cells that are genetically engineered with the recombinant vectors, and the production of at least one antibody construct of the invention by recombinant techniques, as is well known in the art. (See, e.g., Sambrook, et al., supra; Ausubel, et al., supra, each entirely incorporated herein by reference.)

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but not limited to, methotrexate (MTX), dihydrofolate reductase (DHFR, U.S. Pat. Nos. 4,399,216; 4,634,665; 4,656,134; 4,956,288; 5,149,636; 5,179,017, ampicillin, neomycin (G418), mycophenolic acid, or glutamine synthetase (GS, U.S. Pat. Nos. 5,122,464; 5,770,359; 5,827,739) resistance for eukaryotic cell culture, and tetracycline or ampicillin resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. (Such methods are described in the art: Sambrook, supra, Chapters 1-4 and 16-18; Ausubel, supra, Chapters 1, 9, 13, 15, 16.)

At least one antibody of the present invention can be expressed in a modified form, such as a fusion protein, and can include not only secretion signals, but also additional heterologous functional regions. For instance, a region of additional amino acids, particularly charged amino acids, can be added to the N-terminus of an antibody to improve stability and persistence in the host cell, during purification, or during subsequent handling and storage. Also, peptide moieties can be added to an antibody of the present invention to facilitate purification. Such regions can be removed prior to final preparation of an antibody or at least one fragment thereof. (Such methods are described in many standard laboratory manuals: Sambrook, supra; Chapters 17.29-17.42 and 18.1-18.74; Ausubel, supra, Chapters 16, 17 and 18.)

Those of ordinary skill in the art are knowledgeable in the numerous expression systems available for expression of a nucleic acid encoding a protein of the present invention. Alternatively, nucleic acids of the present invention can be expressed in a host cell by turning on (by manipulation) in a host cell that contains endogenous DNA encoding an antibody of the present invention. (Such methods are well known in the art, e.g., as described in U.S. Pat. Nos. 5,580,734, 5,641,670, 5,733,746, and 5,733,761, entirely incorporated herein by reference.)

Illustrative of cell cultures useful for the production of the antibodies, specified portions or variants thereof, are mammalian cells. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. (www.atcc.org). Preferred host cells include cells of lymphoid origin such as myeloma and lymphoma cells. Particularly preferred host cells are P3X63Ag8.653 cells (ATCC Accession Number CRL-1580) and SP2/0-Ag14 cells (ATCC Accession Number CRL-1851). In a particularly preferred embodiment, the recombinant cell is a P3X63Ab8.653 or a SP2/0-Ag14 cell.

Expression vectors for these cells can include one or more of the following expression control sequences, such as, but not limited to an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human immunoglobulin promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra.) Other cells useful for production of nucleic acids or proteins of the present invention are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague, et al., J. Virol. 45:773-781 1983). Additionally, gene sequences to control replication in the host cell can be incorporated into the vector, as known in the art.

J. Cloning and Expression of Antibody Constructs in Mammalian Cells

A typical mammalian expression vector contains at least one promoter element, which mediates the initiation of transcription of mRNA, the antibody coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRS) from Retroviruses, e.g., RSV, HTLVI, HIVI and the early promoter of the cytomegalovirus (CMV). However, cellular elements can also be used (e.g., the human actin promoter). Suitable expression vectors for use in practicing the present invention include, for example, vectors such as pIRES1neo, pRetro-Off, pRetro-On, PLXSN, or pLNCX (Clonetech Lantibodies, Palo Alto, Calif.), pcDNA3.1 (+/−), pcDNA/Zeo (+/−) or pcDNA3.1/Hygro (+/−) (Invitrogen), PSVL and PMSG (Pharmacia, Uppsala, Sweden), pRSVcat (ATCC 37152), pSV2dhfr (ATCC 37146) and pBC12MI (ATCC 67109). Mammalian host cells that could be used include human Hela 293, H9 and Jurkat cells, mouse NIH3T3 and C127 cells, Cos 1, Cos 7 and CV 1, quail QC1-3 cells, mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, or hygromycin allows the identification and isolation of the transfected cells.

The transfected gene can also be amplified to express large amounts of the encoded antibody. The DHFR (dihydrofolate reductase) marker is useful to develop cell lines that carry several hundred or even several thousand copies of the gene of interest. Another useful selection marker is the enzyme glutamine synthase (GS) (Murphy, et al., Biochem. J. 227:277-279 1991; Bebbington, et al., Bio/Technology 10:169-175 1992). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of antibodies.

The expression vectors pC1 and pC4 contain the strong promoter (LTR) of the Rous Sarcoma Virus (Cullen, et al., Molec. Cell. Biol. 5:438-447 1985) plus a fragment of the CMV-enhancer (Boshart, et al., Cell 41:521-530 1985). Multiple cloning sites, e.g., with the restriction enzyme cleavage sites BamHI, XbaI and Asp718, facilitate the cloning of the gene of interest. The vectors contain in addition the 3′ intron, the polyadenylation and termination signal of the rat preproinsulin gene.

K. Cloning and Expression in CHO Cells

The vector pC4 may be used used for the expression of the antibody construct. Plasmid pC4 is a derivative of the plasmid pSV2-dhfr (ATCC Accession No. 37146). The plasmid contains the mouse DHFR gene under control of the SV40 early promoter. Chinese hamster ovary or other cells lacking dihydrofolate activity that are transfected with these plasmids can be selected by growing the cells in a selective medium (e.g., alpha minus MEM, Life Technologies, Gaithersburg, Md.) supplemented with the chemotherapeutic agent methotrexate. The amplification of the DHFR genes in cells resistant to methotrexate (MTX) has been well documented (see, e.g., F. W. Alt, et al., J. Biol. Chem. 253:1357-1370 1978; J. L. Hamlin and C. Ma, Biochem. et Biophys. Acta 1097:107-143 1990; and M. J. Page and M. A. Sydenham, Biotechnology 9:64-68 1991). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach can be used to develop cell lines carrying more than 1,000 copies of the amplified gene(s). Subsequently, when the methotrexate is withdrawn, cell lines are obtained that contain the amplified gene integrated into one or more chromosome(s) of the host cell.

Plasmid pC4 contains for expressing the gene of interest the strong promoter of the long terminal repeat (LTR) of the Rous Sarcoma Virus (Cullen, et al., Molec. Cell. Biol. 5:438-447 1985) plus a fragment isolated from the enhancer of the immediate early gene of human cytomegalovirus (CMV) (Boshart, et al., Cell 41:521-530 1985). Downstream of the promoter are BamHI, XbaI, and Asp718 restriction enzyme cleavage sites that allow integration of the genes. Behind these cloning sites the plasmid contains the 3′ intron and polyadenylation site of the rat preproinsulin gene. Other high efficiency promoters can also be used for the expression, e.g., the human b-actin promoter, the SV40 early or late promoters or the long terminal repeats from other retroviruses, e.g., HIV and HTLVI. Clontech's Tet-Off and Tet-On gene expression systems and similar systems can be used to express the Modified antibody construct in a regulated way in mammalian cells (M. Gossen, and H. Bujard, Proc. Natl. Acad. Sci. USA 89: 5547-5551 1992). For the polyadenylation of the mRNA other signals, e.g., from the human growth hormone or globin, genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 is digested with restriction enzymes and then dephosphorylated using calf intestinal phosphatase by procedures known in the art. The vector is then isolated from a 1% agarose gel.

The DNA sequence encoding the complete Modified antibody construct is used, e.g., as presented in SEQ ID NOS: 7, and 8, corresponding to HC and LC variable regions of a Modified antibody construct of the present invention, according to known method steps. Isolated nucleic acid encoding a suitable human constant region (i.e., HC and LC regions) is also used in this construct.

The isolated variable and constant region encoding DNA and the dephosphorylated vector are then ligated with T4 DNA ligase. E. coli HB101 or XL-1 Blue cells are then transformed and bacteria are identified that contain the fragment inserted into plasmid pC4 using, for instance, restriction enzyme analysis.

Chinese hamster ovary (CHO) cells lacking an active DHFR gene are used for transfection. 5 μg of the expression plasmid pC4 is cotransfected with 0.5 □g of the plasmid pSV2-neo using lipofectin. The plasmid pSV2neo contains a dominant selectable marker, the neo-gene from Tn5 encoding an enzyme that confers resistance to a group of antibiotics including G418. The cells are seeded in α minus MEM supplemented with 1 μg /ml G418. After 2 days, the cells are trypsinized and seeded in hybridoma cloning plates (Greiner, Germany) in α minus MEM supplemented with 10, 25, or 50 ng/ml of methotrexate plus 1 μg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 mM, 2 mM, 5 mM, 10 mM, 20 mM). The same procedure is repeated until clones are obtained that grow at a concentration of 100-200 mM. Expression of the desired gene product is analyzed, for instance, by SDS-PAGE and Western blot or by reverse phase HPLC analysis.

L. Purification of an Antibody

A modified antibody construct can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. (See, e.g., Colligan, Current Protocols in Immunology, or Current Protocols in Protein Science, John Wiley & Sons, NY, N.Y., 1997-2001, Chapters 1, 4, 6, 8, 9, 10, each entirely incorporated herein by reference.)

J. Utility

The isolated nucleic acids of the present invention can be used for production of at least one antibody construct or specified variant thereof, which can be used to measure an effect in a cell, tissue, organ or animal (including mammals and humans), to diagnose, monitor, modulate, treat, alleviate, help prevent the incidence of, or reduce the symptoms of, at least one condition, selected from, but not limited to, at least one of an immune disorder or disease, a cardiovascular disorder or disease, an infectious, malignant, and/or neurologic disorder or disease, an allergic disorder or disease; a skin disorder or disease; a hematological disorder or disease, and/or a pulmonary disorder or disease, or other known or specified condition.

Such a method can comprise administering an effective amount of a composition or a pharmaceutical composition comprising at least one modified antibody construct to a cell, tissue, organ, animal or patient in need of such modulation, treatment, alleviation, prevention, or reduction in symptoms, effects or mechanisms. The effective amount can comprise an amount of about 0.001 to 500 mg/kg per single (e.g., bolus), multiple or continuous administration, or to achieve a serum concentration of 0.01-5000 μg/ml serum concentration per single, multiple, or continuous adminstration, or any effective range or value therein, as done and determined using known methods, as described herein or known in the relevant arts.

K. Modified Antibody Construct Compositions

The present invention also provides at least one antibody construct composition comprising at least one, at least two, at least three, at least four, at least five, at least six or more antibodies thereof, as described herein and/or as known in the art that are provided in a non-naturally occurring composition, mixture or form. Such compositions comprise non-naturally occurring compositions comprising at least one modified antibody of the invention in combination with a pharmaceutically acceptable carrier. Such antibody construct compositions can include anywhere from 40-99% of the modified antibody construct of the invention. Such composition percentages are by weight, volume, concentration, molarity, or molality as liquid or dry solutions, mixtures, suspension, emulsions or colloids, as known in the art or as described herein.

Modified antibody constructs or specified portion or variant compositions of the present invention can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art; (Gennaro, Ed., Remington's Pharmaceutical Sciences, 18^{th} Edition, Mack Publishing Co. Easton, Pa. 1990.) Pharmaceutically acceptable carriers can be routinely selected that are suitable for the mode of administration, solubility and/or stability of the Modified antibody construct composition as well known in the art or as described herein.

Pharmaceutical excipients and additives useful in the present composition include but are not limited to proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/Modified antibody construct or specified portion or variant components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Carbohydrate excipients suitable for use in the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present invention are mannitol, trehalose, and raffinose.

Modified antibody construct compositions can also include a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers for use in the present compositions are organic acid salts such as citrate.

Additionally, the modified antibody construct or specified portion or variant compositions of the invention can include polymeric excipients/additives such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the Modified antibody construct compositions according to the invention are known in the art, (e.g., as listed in Remington: The Science & Practice of Pharmacy, 19^{th} ed., Williams & Williams, 1995; Physician's Desk Reference, 52^{nd} ed., Medical Economics, Montvale, N.J. 1998 the disclosures of which are entirely incorporated herein by reference.) Preferrred carrier or excipient materials are carbohydrates (e.g., saccharides and alditols) and buffers (e.g., citrate) or polymeric agents.

L. Formulations

As noted above, the invention provides for stable formulations, which is preferably a phosphate buffer with saline or a chosen salt, as well as preserved solutions and formulations containing a preservative as well as multi-use preserved formulations suitable for pharmaceutical or veterinary use, comprising at least one modified antibody construct in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4., 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1., 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

As noted above, the invention provides an article of manufacture, comprising packaging material and at least one vial comprising a solution of at least one modified antibody construct with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further comprises an article of manufacture, comprising packaging material, a first vial comprising lyophilized at least one Modified antibody construct, and a second vial comprising an aqueous diluent of prescribed buffer or preservative, wherein said packaging material comprises a label that instructs a patient to reconstitute the at least one modified antibody construct in the aqueous diluent to form a solution that can be held over a period of twenty-four hours or greater.

The range of at least one modified antibody construct in the product of the present invention includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

Preferably, the aqueous diluent optionally further comprises a pharmaceutically acceptable preservative. Preferred preservatives include those selected from the group consisting of phenol, m-cresol, p-cresol, o-cresol, chlorocresol, benzyl alcohol, alkylparaben (methyl, ethyl, propyl, butyl and the like), benzalkonium chloride, benzethonium chloride, sodium dehydroacetate and thimerosal, or mixtures thereof. The concentration of preservative used in the formulation is a concentration sufficient to yield an anti-microbial effect. Such concentrations are dependent on the preservative selected and are readily determined by the skilled artisan.

Other excipients, e.g. isotonicity agents, buffers, antioxidants, preservative enhancers, can be optionally and preferably added to the diluent. An isotonicity agent, such as glycerin, is commonly used at known concentrations. A physiologically tolerated buffer is preferably added to provide improved pH control. The formulations can cover a wide range of pHs, such as from about pH 4 to about pH 10, and preferred ranges from about pH 5 to about pH 9, and a most preferred range of about 6.0 to about 8.0. Preferably the formulations of the present invention have pH between about 6.8 and about 7.8. Preferred buffers include phosphate buffers, most preferably sodium phosphate, particularly phosphate buffered saline (PBS).

Other additives, such as a pharmaceutically acceptable solubilizers like Tween 20 (polyoxyethylene (20) sorbitan monolaurate), TWEEN 40 (polyoxyethylene (20) sorbitan monopalmitate), TWEEN 80 (polyoxyethylene (20) sorbitan monooleate), Pluronic F68 (polyoxyethylene polyoxypropylene block copolymers), and PEG (polyethylene glycol) or non-ionic surfactants such as polysorbate 20 or 80 or poloxamer 184 or 188, Pluronic® polyls, other block co-polymers, and chelators such as EDTA and EGTA can optionally be added to the formulations or compositions to reduce aggregation. These additives are particularly useful if a pump or plastic container is used to administer the formulation. The presence of pharmaceutically acceptable surfactant mitigates the propensity for the protein to aggregate.

The formulations of the present invention can be prepared by a process which comprises mixing at least one antibody construct and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing the at least one antibody construct and preservative in an aqueous diluent is carried out using conventional dissolution and mixing procedures. To prepare a suitable formulation, for example, a measured amount of at least one antibody construct in buffered solution is combined with the desired preservative in a buffered solution in quantities sufficient to provide the protein and preservative at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed formulations can be provided to patients as clear solutions or as dual vials comprising a vial of lyophilized at least one antibody construct that is reconstituted with a second vial containing water, a preservative and/or excipients, preferably a phosphate buffer and/or saline and a chosen salt, in an aqueous diluent. Either a single solution vial or dual vial requiring reconstitution can be reused multiple times and can suffice for a single or multiple cycles of patient treatment and thus can provide a more convenient treatment regimen than currently available.

The present claimed articles of manufacture are useful for administration over a period of immediately to twenty-four hours or greater. Accordingly, the presently claimed articles of manufacture offer significant advantages to the patient. Formulations of the invention can optionally be safely stored at temperatures of from about 2° C. to about 40° C. and retain the biologically activity of the protein for extended periods of time, thus, allowing a package label indicating that the solution can be held and/or used over a period of 6, 12, 18, 24, 36, 48, 72, or 96 hours or greater. If preserved diluent is used, such label can include use up to 1-12 months, one-half, one and a half, and/or two years.

The solutions of at least one modified antibody construct in the invention can be prepared by a process that comprises mixing at least one antibody in an aqueous diluent. Mixing is carried out using conventional dissolution and mixing procedures. To prepare a suitable diluent, for example, a measured amount of at least one antibody in water or buffer is combined in quantities sufficient to provide the protein and optionally a preservative or buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed products can be provided to patients as clear solutions or as dual vials comprising a vial of lyophilized at least one anti-Modified antibody construct that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available.

The claimed products can be provided indirectly to patients by providing to pharmacies, clinics, or other such institutions and facilities, clear solutions or dual vials comprising a vial of lyophilized at least one Modified antibody construct that is reconstituted with a second vial containing the aqueous diluent. The clear solution in this case can be up to one liter or even larger in size, providing a large reservoir from which smaller portions of the at least one antibody solution can be retrieved one or multiple times for transfer into smaller vials and provided by the pharmacy or clinic to their customers and/or patients.

Recognized devices comprising these single vial systems include those pen-injector devices for delivery of a solution such as BD Pens, BD Autojector®, Humaject® NovoPen®, B-D®Pen, AutoPen®, and OptiPen®, GenotropinPen®, Genotronorm Pen®, Humatro Pen®, Reco Pen®, Roferon Pen®, Biojector®, iject®, J-tip Needle-Free Injector®, Intraject®, Medi-Ject®, e.g., as made or developed by Becton Dickensen (Franklin Lakes, N.J., www.bectondickenson.com), Disetronic (Burgdorf, Switzerland, www.disetronic.com; Bioject, Portland, Oreg. (www.bioject.com); National Medical Products, Weston Medical (Peterborough, UK, www.weston-medical.com), Medi-Ject Corp (Minneapolis, Minn., www.mediject.com). Recognized devices comprising a dual vial system include those pen-injector systems for reconstituting a lyophilized drug in a cartridge for delivery of the reconstituted solution such as the HumatroPen®.

The products presently claimed include packaging material. The packaging material provides, in addition to the information required by the regulatory agencies, the conditions under which the product can be used. The packaging material of the present invention provides instructions to the patient to reconstitute the at least one anti-Modified antibody construct in the aqueous diluent to form a solution and to use the solution over a period of 2-24 hours or greater for the two vial, wet/dry, product. For the single vial, solution product, the label indicates that such solution can be used over a period of 2-24 hours or greater. The presently claimed products are useful for human pharmaceutical product use.

The formulations of the present invention can be prepared by a process that comprises mixing at least one modified antibody construct and a selected buffer, preferably a phosphate buffer containing saline or a chosen salt. Mixing the at least one antibody and buffer in an aqueous diluent is carried out using conventional dissolution and mixing procedures. To prepare a suitable formulation, for example, a measured amount of at least one antibody in water or buffer is combined with the desired buffering agent in water in quantities sufficient to provide the protein and buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed stable or preserved formulations can be provided to patients as clear solutions or as dual vials comprising a vial of lyophilized at least one anti-Modified antibody construct that is reconstituted with a second vial containing a preservative or buffer and excipients in an aqueous diluent. Either a single solution vial or dual vial requiring reconstitution can be reused multiple times and can suffice for a single or multiple cycles of patient treatment and thus provides a more convenient treatment regimen than currently available.

At least one modified antibody construct in either the stable or preserved formulations or solutions described herein, can be administered to a patient in accordance with the present invention via a variety of delivery methods including SC or IM injection; transdermal, pulmonary, transmucosal, implant, osmotic pump, cartridge, micro pump, or other means appreciated by the skilled artisan, as well-known in the art.

M. Therapeutic Applications

The present invention also provides a method for modulating or treating a disease, in a cell, tissue, organ, animal, or patient, as known in the art or as described herein, using at least one modified antibody construct of the present invention.

The present invention also provides a method for modulating or treating at least one disease, in a cell, tissue, organ, animal, or patient including, but not limited to, at least one of obesity, an immune related disease, a cardiovascular disease, an infectious disease, a malignant disease or a neurologic disease.

Typically, treatment of pathologic conditions is effected by administering an effective amount or dosage of at least one modified antibody construct composition that total, on average, a range from at least about 0.01 to 500 milligrams of at least one anti-Santibody per kilogram of patient per dose, and preferably from at least about 0.1 to 100 milligrammodified antibody construct/kilogram of patient per single or multiple administration, depending upon the specific activity of contained in the composition. Alternatively, the effective serum concentration can comprise 0.1-5000 ug/ml serum concentration per single or multiple adminstration. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

Preferred doses can optionally include 0.1-100 mg/kg/administration, or any range, value or fraction thereof, or to achieve a serum concentration of 0.1-5000 μg/ml serum concentration per single or multiple administration, or any range, value or fraction thereof.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and preferably 0.1 to 10 milligrams per kilogram per administration or in sustained release form is effective to obtain desired results.

Dosage forms (composition) suitable for internal administration generally contain from about 0.1 milligram to about 500 milligrams of active ingredient per unit or container. In these pharmaceutical compositions the active ingredient will ordinarily be present in an amount of about 0.5-99.999% by weight based on the total weight of the composition.

For parenteral administration, the antibody can be formulated as a solution, suspension, emulsion or lyophilized powder in association, or separately provided, with a pharmaceutically acceptable parenteral vehicle. Examples of such vehicles are water, saline, Ringer's solution, dextrose solution, and 1-10% human serum albumin. Liposomes and nonaqueous vehicles such as fixed oils can also be used. The vehicle or lyophilized powder can contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

Having generally described the invention, the same will be more readily understood by reference to the following examples, which are provided by way of illustration and are not intended as limiting.

### EXAMPLE 1

### Preparation of Modified Antibody

Genes encoding the modified Ab were prepared by using PCR amplification and recombinant DNA methods to substitute a) DNA sequence encoding the V_{H} and C_{H}1 domains of an IgG1,k Ab with DNA sequence encoding the V_{L} and C_{L} domains of the same Ab, and b) DNA sequence encoding the V_{L} and C_{L} domains of the same Ab with DNA sequence encoding the V_{H} and C_{H}1 domains of the same Ab.

To prepare a plasmid encoding a V_{L}-C_{L}-hg-C_{H}2—C_{H}3 HC, plasmid p3123 containing the original LC cDNA sequence was used as template in an overlapping PCR protocol that amplified a 765 bp fragment extending from the ATG translation start codon through the signal sequence, V, and C_{L} domains. The downstream oligonucleotide used to prime the reaction further served to replace the naturally-occurring translation stop codon immediately following the Cₖ coding sequence with DNA sequence encoding the first 23 bases of the IgG1 HC hinge sequence. Plasmid p3122 containing the original HC cDNA sequence was then used in a second PCR reaction that amplified a 715 bp fragment that included the last 22 bases of the C_{L} coding sequence (incorporated into the upstream oligonucldotide primer) followed by sequences encoding the hinge, C_{H}2, and C_{H}3 domains. The downstream oligonucleotide primer provided a NotI restriction site. The two PCR products were effectively joined in a third PCR reaction that contained the 765 bp and 715 bp amplified products, the upstream oligonucleotide primer from the first PCR reaction, and the downstream oligonucleotide primer from the second PCR reaction. Amplification therefore depended on the two fragments cross-annealing at the 22 bp of overlapping sequence that they both contained. The resulting amplified product was 1480 bp in length and encoded V_{L}-C_{L}-hg-C_{H}2-C_{H}3, flanked by XbaI and NotI restriction sites, respectively. The amplified DNA was digested with XbaI and NotI and cloned between the XbaI and NotI sites of vector, p2106, to form the final expression plasmid, p4034, encoding the new HC. Vector p2106 provides the CMV immediate early promoter and intron A upstream of the ATG start codon, and SV40-derived transcription termination sequence.

To prepare a plasmid encoding a V_{H}-C_{H}1 LC, plasmid p3122 containing the original HC cDNA sequence was used as template in an oligonucleotide-primed PCR reaction that amplified an 808 bp fragment extending from the ATG translation start codon through the signal sequence, V, and C_{H}1 domains. The amplified product contained a translation stop codon immediately after the DKKV amino acid sequence at the C-terminal end of the C_{H}1 domain. The oligonucleotide primers also provided, for cloning purposes, an XbaI restriction site upstream of the ATG start codon and a NotI restriction site downstream of the stop codon. The amplified DNA was digested with XbaI and NotI and the cleaved DNA cloned between the XbaI and NotI sites of plasmid vector, p2106, to form the final expression plasmid, p4033, encoding the new LC.

To prepare modified Ab by co-expression of the new HC and new LC plasmids, human HEK 293 cells were transiently transfected with plasmids p4033 and p4034 by lipofection. Briefly, the day before transfection, cells grown in DMEM with 10% FBS were plated in a six-well plate and incubated overnight at 37° C. in a 5% CO₂ incubator. The next day, 1 μg each of p4033 and p4034 resuspended in 100 μl of serum-free medium (Opti-MEM I) was mixed with 10 μl of lipofection reagent and allowed to sit at room temperature for 10 minutes. Medium was aspirated from the cells and fresh DMEM with 10% FBS was added. After incubation, the DNA/lipofection reagent complexes were added to the cells and gently swirled to mix. After a further incubation at 37° C. in a 5% CO₂ incubator for 72 hours to allow time for antibody expression and secretion, the cell supernatant was collected and centrifuged to remove cellular debris.

To test for the presence of modified Ab in the cell supernatant, ELISA assays using four different capture reagents were performed. 96-well EIA plates were coated with either a) goat polyclonal anti-human IgG Fc fragment Abs, b) C508 monoclonal Ab specific for the original Ab V region, c) C585 monoclonal Ab specific for the idiotype portion (antigen-binding) of the original Ab V region, and d) a negative control monoclonal Ab of the same isotype as C508 and C585 (mouse IgG2bk). The coatings were performed in 0.05 M carbonate buffer, pH 9.5 and incubated overnight at 4° C. Plates were blocked using 1% BSA in PBS. Cell supernatant from the HEK 293 cell transient transfections was serially diluted and added to the blocked plates. Control samples included cell supernatant from HEK 293 cells transfected at the same time with plasmids p3122 and p3123 encoding the original, unmodified Ab, and the purified original Ab used to prepare a standard curve. Captured protein was detected using an HRP-labeled goat anti-human Fc-gamma antibody and a TMB substrate. Color development reactions were stopped with 0.5M HCl. Plates were read at 450 nm.

A positive ELISA signal obtained when anti-human IgG Fc fragment Abs were used as capture reagent revealed that an Ab-like molecule was indeed present in the supernatant of cells that had been transfected with the modified HC and modified LC. Because HCs are normally not secreted from cells unless they are associated with LCs, this result alone made it likely that the modified HC and LC were associating with each other to form the (HL)₂ four-chain complex typical of IgG Abs. It also showed that the epitopes on the Fc domain of the modified Ab were well-recognized by the goat anti-human Fc Abs. Using the assumption that the goat anti-human Fc Abs reacted with the modified Ab and unmodified Ab equally well, calculations from the ELISA data using the original Ab standard curve showed expression levels of the modified Ab (˜2.7 μg/ml) to be similar to expression levels of the unmodified Ab (˜2.0 μg/ml).

Using the concentrations derived from the unmodified Ab standard, binding of the modified and unmodified Ab samples to the anti-V region Abs C508 and C585 was plotted against the protein concentrations. The results showed that binding by the modified and unmodified Abs were found to be indistinguishable. This suggests that the V region binding sites for C508 and C585 monclonal Abs may have been completely preserved in the modified Ab.

Oligonucleotide Primers Used to Prepare Modified Ab Genes—

| | |
|---|---|
| New LC 5′ | 5′-TGTCTAGAAGCTGGGTAC-3′ |
| | XbaI |
| New LC 3′ | 5′-AAGCGGCCGCCTAAACTTTCTTGTCCACCTTGGTG-3′ |
| | NotI |
| New HC 5′ | 5′-TGTCTAGAAGCTGGGTAC-3′ |
| | XbaI |
| New LC Overlap Sense | 5′-GAGCTTCAACAGGGGAGAGTGTGAGCCCAAATCTTGTGACAAAAC-3′-3′ |
| | 3′ END OF LC 5′ END OF HINGE |
| New LC Overlap Antisense | 5′-GTTTTGTCACAAGATTTGGGCTCACACTCTCCCCTGTTGAAGCTC-3′ |
| | 5′ END OF HINGE 3′ END OF LC |

What is claimed is:

1. A heterodimeric protein binding composition comprising a modified immunoglobulin molecule having a heavy and light chain, wherein one or more light chain domains are substituted for one or more heavy chain domains on the immunoglobulin heavy chain, and/or one or more heavy chain domains are substituted for one or more light chain domains on the immunoglobulin light chain of the immunoglobulin molecule.

2. The modified immunoglobulin molecule of claim 1, wherein the V_{L}-C_{L} light chain domains are substituted for the V_{H}-C_{H}1 regions of the immunoglobulin heavy chain and the V_{H}-C_{H}1 domains of the heavy chain are substituted for the V_{L}-C_{L} light chain regions of the immunoglobulin light chain.

3. The modified immunoglobulin molecule of claim 1, wherein the immunoglobulin molecule is IgG1.

4. The modified immunoglobulin molecule of claim 1 further comprising an antigen-binding region.

5. The modified immunoglobulin molecule of claim 1, wherein the immunoglobulin molecule is an IgA, IgG, IgM, IgE, or IgD molecule.

6. A polynucleotide that encodes a modified immunoglobulin molecule of claim 1.

7. A vector comprising the polynucleotide of claim 6.

8. A host cell transfected with the vector of claim 8.

9. A method of producing a modified immunoglobulin molecule comprising culturing the host cell of claim 8 and recovering the modified immunoglobulin molecule so produced.

10. The method of claim 9, wherein the cell is a eucaryotic or procaryotic cell.

11. The method of claim 10, wherein the cell is a mammalian, avian, reptilian, insect, plant, bacterial, fungal or yeast cell.

12. The method of claim 11, wherein the mammalian cell is a human, rabbit, murine, rat, hamster or bovine cell.

13. The method of claim 12, wherein the host cell is at least one selected from COS-1, COS-7, HEK 293, BHK21, CHO, BSC-1, HepG2, 653, SP2/0, NS/0, HeLa, other myeloma cells or lymphoma cells, or any derivative, immortalized or transformed cell thereof.

14. A pharmaceutical composition comprising the modified immunoglobulin molecule of claim 1 and a pharmaceutically acceptable carrier.

15. A method of treating or protecting against an infection in a subject comprising administering the composition of claim 14 to the subject.

16. A nucleic acid composition, comprising an isolated nucleic acid according to claim 6 and a carrier or diluent.

17. An antibody vector according to claim 7, wherein said vector comprises at least one promoter selected from the group consisting of a late or early SV490 promoter, a CMV promoter, an HSV tk promoter, a pgk (phosphoglycerate kinase) promoter, a human immunoglobulin promoter or an EF-1 alpha promoter.

18. An antibody vector according to claim 7, wherein said vector comprises at least one selection gene or portion thereof selected from at least one of methotrexate (MTX), green fluorescent protein (GFP), dihydrofolate reductase (DHFR), neomycin (G418), or glutamine synthetase (GS).

19. A method for producing a modified immunoglobulin of claim 1 comprising translating a nucleic acid according to claim 6 or an endogenous nucleic acid that hybridizes thereto under stringent conditions, under conditions in vitro, in vivo or situ, such that the modified immunoglobulin is expressed in detectable or recoverable amounts.

20. A method for modulating at least one disorder or condition in a cell, tissue, organ or animal, comprising contacting or administering a disorder or condition modulating effective amount of at least one modified immunoglobulin according to claim 1 with, or to, said cell, tissue, organ or animal.

21. A method according to claim 20 wherein said effective amount is 0.01-100 mg/kilogram of said cells, tissue, organ or animal.

22. A method according to any of claims 20-21, wherein said contacting or said administrating is by at least one mode selected from intravenous, intramuscular, colus, subcutaneous, respiratory, inhalation, vaginal, rectal, buccal, sublingual, intranasal, or transdermal.

23. A formulation comprising at least one modified immunoglobulin according to claim 1, and at least one selected from sterile water, sterile buffered water, or at least one preservative selected from the group consisting of phenol, m-cresol, p-cresol, o-cresol, chlorocresol, benzyl alcohol, phenylmercuric nitrite, phenoxyethanol, formaldehyde, chlorobutanol, magnesium chloride, alkylparaben, benzalkonium chloride, benzethonium chloride, sodium dehydroacetate and thimerosal, ormixtures thereof, in an aqueous diluent.

24. A formulation of claim 23, wherein the concentration of modified immunoglobulin is about 0.1 mg/ml to about 100 mg/ml.

25. A formulation of claim 24, further comprising an isotonicity agent.

26. A formulation of claim 25, further comprising a physiologically acceptable buffer.

27. A formulation comprising at least one modified immunoglobulin according to claim 1 in lyophilized form in a first container, and an optional second container comprising sterile water, sterile buffered water, or at least one preservative selected from the group consisting of phenol, m-cresol, p-cresol, o-cresol, chlorocresol, benzyl alcohol, phenylmercuric nitrite, phenoxyethanol, formaldehyde, chlorobutanol, magnesium chloride, alkylparaben, benzalkonium chloride, benzethonium chloride, sodium dehydroacetate and thimerosal, or mixtures thereof in an aqueous diluent.

28. A method of treating a disease or condition in a patient, comprising administering to a patient in need thereof a formulation according to claim 24.

29. A method for producing at least one modified immunoglobulin according to claim 1, comprising providing a host cell or transgenic animal or transgenic plant or plant cell capable of expressing in recoverable amounts said antibody or specified portion or variant.

30. A method according to claim 29, wherein said host cell is a mammalian cell, a plant cell or a yeast cell.

31. A method according to claim 30, wherein said transgenic animal is a mammal.

32. A method according to claim 33, wherein said transgenic mammal is selected from a goat, a cow, a sheep, a horse, and a non-human primate.

33. A transgenic animal or plant expressing at least one antibody according to claim 1.

34. At least one modified immunoglobulin produced by a method according to claim 29.

35. A method of modifying the ability of an immunoglobulin molecule having FcR-binding and C1q-binding domains to recruit effector functions such as ADCC, FcR-mediated phagocytosis, and complement lysis, which method comprises substituting one or more light chain domains for one or more heavy chain domains on the immunoglobulin heavy chain, and/or substituting one or more heavy chain domains for one or more light chain domains on the immunoglobulin light chain of the immunoglobulin molecule thereby reorienting the relative position of the FcR-binding and C1q-binding domains relative to the antigen-binding domain of the immunoglobulin molecule.
